(19) United States
(12) Patent Application Publication
(10) Pub. No.: US 2006/0286078 A1
(43) Pub. Date: Dec. 21, 2006

(54) Methods of treating cardiorenal syndrome and hepatorenal syndrome

(76) Inventor: H. David Humes, Ann Arbor, MI (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/452,089

(22) Filed: Jun. 13, 2006

Related U.S. Application Data
(60) Provisional application No. 60/690,521, filed on Jun. 15, 2005.

(51) Int. Cl. A61K 35/23; B01D 61/02

(52) U.S. Cl. 424/93.7; 210/646

(57) ABSTRACT

A method of treating a patient with cardiorenal syndrome and hepatorenal syndrome are provided in which a portion of the body fluid of the patient is exposed to renal epithelial cells, outside of the kidney of the patient, whereby the body fluid is in fluid communication with renal epithelial cells and is modified by renal epithelial cells.

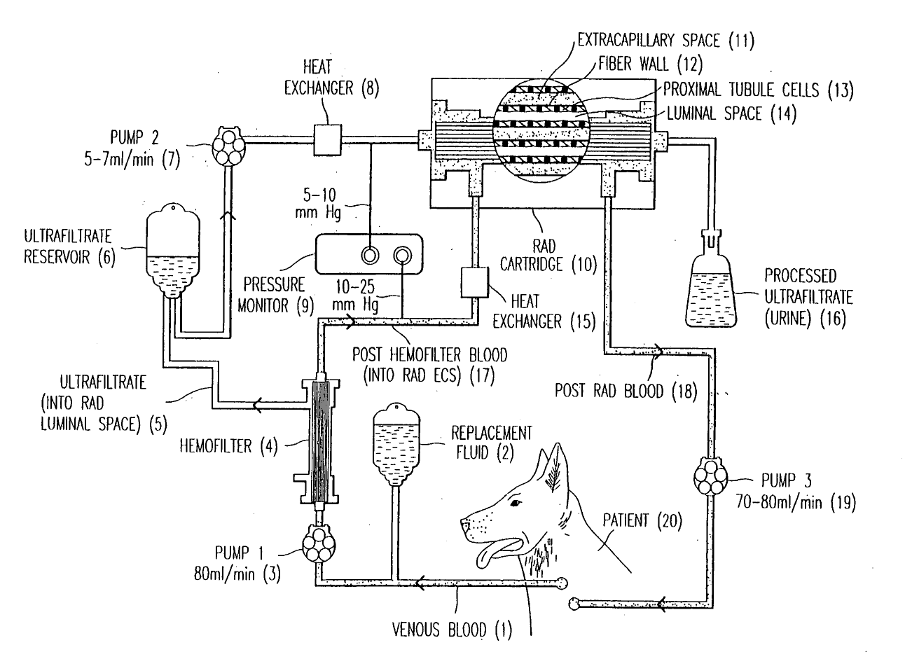

### RELATED APPLICATION(S)

This application claims the benefit of U.S. Provisional Application No. 60/690,521, filed on Jun. 15, 2005. The entire teachings of the above application(s) are incorporated herein by reference.

### BACKGROUND OF THE INVENTION

1. Field of the Invention

The present invention relates to methods of treating cardiorenal syndrome and hepatorenal syndrome.

2. Description of the Background

Cardiorenal syndrome is a condition in which patients with congestive heart failure (class ¾) also suffer from concomitant renal dysfunction. This condition is also characterized by diuretic resistance and progressive fluid overload. The cause of the syndrome is not presently known and there is no consensus for the appropriate management of patients afflicted with this condition. It is expected that cardiorenal syndrome will become more common in the future because patients with heart failure are surviving longer and dying less frequently from primary arrhythmias.

Hepatorenal syndrome is a condition that occurs in patients with liver diseases. This condition is characterized by impaired renal function and marked abnormalities in the arterial circulation and activity of the endogenous vasoactive systems. In the kidney, there is marked renal vasoconstriction that results in low glomerular filtration rate. In the extrarenal circulation, there is predominance of arterial vasodilation that results in reduction of total systemic vascular resistance and arterial hypotension. It is estimated that about 40% of patients with cirrhosis and ascites will develop hepatorenal syndrome during the course of their disease. Patients with hepatorenal syndrome have very short survival time.

Therefore, there remains a need for new methods of treating cardiorenal syndrome and hepatorenal syndrome.

### SUMMARY OF THE INVENTION

It is an object of the present invention to provide methods of treating patients with cardiorenal syndrome and hepatorenal syndrome.

This object of the present invention and others may be accomplished through methods of treatment comprising exposing at least a portion of the body fluid of the patient to renal epithelial cells, outside of the kidney of the patient, whereby the body fluid comes into fluid communication with renal epithelial cells and is modified by renal epithelial cells.

In a preferred embodiment of the invention, a portion of the body fluid is removed from the patient, the removed portion of the body fluid is exposed to the renal epithelial cells, whereby the body fluid comes into fluid communication with the renal epithelial cells and is modified by renal epithelial cells. The body fluid which has been modified by the renal epithelial cells is then returned to the patient.

### BRIEF DESCRIPTION OF THE FIGURE

The FIGURE is a diagram depicting treatment of cardiorenal syndrome or hepatorenal syndrome with the RAD.

### DETAILED DESCRIPTION OF THE INVENTION

The present invention is based, in part, on the inventor's recognition that exposing a body fluid of a patient with cardiorenal syndrome to renal epithelial cells and then returning at least a portion of the treated body fluid to the patient results in amelioration of cardiorenal syndrome. One embodiment of the invention is directed to a method of treating a patient with cardiorenal syndrome comprising exposing at least a portion of the body fluid of the patient to renal epithelial cells, whereby the body fluid comes into communication with renal epithelial cells and therefore is modified by renal epithelial cells. Fluid communication can be achieved by direct contact or through a porous material, such as the membrane of a hollow fiber. As used herein, “modified” means to remove, provide and/or catabolize one or more factors. Without being limited to any particular theory, the renal epithelial cells may provide and/or catabolize one or more factors (e.g., hormones, activated vitamin D, renalase, cytokines such as IL-1, IL-6, IL-8, IL-10 and G-CSF, and vasoactive compounds such as renin, angiotensin and kallekreins), which improve cardiac contractility as well as native kidney function with increased urine formation. The renal epithelial cells may also remove toxic factors from the body fluid (e.g. myoglobin and cytokines)

Thus, the patient to be treated according to the present invention is suffering from cardiorenal syndrome as discussed above. The patient may have been diagnosed with cardiorenal syndrome by a healthcare provider according to diagnostic procedures well-known to those skilled in the art. For a discussion of cardiorenal syndrome, see Shlipak, et al., Circulation, 2004, 110, 1514-1517 and Bongartz, et al., European Heart Journal, 2005, 26, 11-17, incorporated herein by reference.

Congestive heart failure, present in cardiorenal syndrome as discussed above, involves the loss of ventricular function, various adaptational responses (neurohormonal activation, peripheral vasoconstriction, and salt and water retention). See Cowburn et al., Eur. Heart J., 1988, 19, 696-710, incorporated herein by reference.

Subjects with cardiorenal syndrome are also generally resistant to diuretics and inotropes, for example Nesiritide. See Wang et al., Circulation, 2004, 110, 1620-1625 and Neuberg et al., American Heart Journal, July 2002, 31-38.

The present invention is also directed to a method of treating a patient with hepatorenal syndrome. The method comprises exposing at least a portion of the body fluid of the patient to renal epithelial cells, outside of the kidney of the patient, whereby the body fluid comes into fluid communication with the renal epithelial cells and therefore is modified by renal epithelial cells. Without being limited to any particular theory, the renal epithelial cells may provide and/or catabolize one or more factors (e.g., hormones, activated vitamin D, renalase, cytokines such as IL-1, IL-6, IL-8, IL-10 and G-CSF, and vasoactive compounds such as renin, angiotensin and kallekreins), which improve vascular resistance and arterial hypotension, diminish renal vascular vasoconstriction and provide improved native kidney function with increased urine formation. The renal epithelial cells may also remove toxic factors from the body fluid (e.g. myoglobin and cytokines).

Thus, the patient to be treated according to the present invention is suffering from hepatorenal syndrome as discussed above. The patient may have been diagnosed with hepatorenal syndrome by a healthcare provider according to diagnostic procedures well-known to those skilled in the art. For a discussion of hepatorenal syndrome, see Epstein, Seminars in Nephrology, 1997, 17, 563-575 and Kramer et al., Seminars in Nephrology, 2002, 22, 290-301, incorporated herein by reference.

As used herein, “a patient” may be a human or a non-human animal, such as a mammal. Exemplary non-human animals include dogs, cats, horses, cows, sheep, goats, and pigs. Thus, the present invention may have use in veterinary and livestock management fields.

As used herein, “renal epithelial cells” refers to cells that are involved in renal tubule function. Renal tubule function may include, for example, immune modulation including effecting the host defense system, providing for antigen presentation and cytokine production, and also metabolic/endocrine functions including the production of hormone, vitamin and vasoactive compounds (such as renin, angiotensin and kallekreins) and helping to maintain calcium and phosphorus homeostasis.

Renal epithelial cells may include renal tubule cells along the various segments of the kidney nephron, including proximal and distal epithelial cells.

An important feature of the present invention is that a body fluid of the patient is in fluid communication with renal epithelial cells. It is important to note that the body fluid of the patient is fluid communication with renal epithelial cells outside of the kidney. In the present invention, the natural flow of the body fluid is interrupted so that the fluid can interact with the renal epithelial cells. The body fluid is then returned to the course of natural flow in the patient's body. Thus, the present invention is distinct from the natural physiological processes which occur in the kidney.

Methods and devices for exposing a body fluid with renal epithelial cells and then returning the treated fluid to the patient are well-known in the art. See, for example, Humes et al., Kidney Int 55:2502-2514, 1999; Humes et al., Nature Biotech 17:451-455, 1999; Humes, Seminars in Nephrology 20:71-82, 2000; MacKay et al., ASAIO Journal 44:179-183, 1998; U.S. Pat. Nos. 6,150,164, 6,561,997 and 5,549,674, all of which are incorporated herein by reference in their entirety.

In a particularly preferred embodiment of the invention, the body fluid of the patient is in fluid communication with the renal epithelial cells in a renal tubule assist device (RAD). As used herein, the term “renal tubule assist device” refers to a device which contains (1) renal epithelial cells and (2) an inlet and outlet for the body fluid, where the body fluid is in fluid communication with the renal epithelial cells inside the device. RAD can also be referred as renal bioreplacement therapy (RBT). Such a device is described in detail in the publications cited immediately above. An example of a suitable RAD is shown in the FIGURE as element (10) in the circuit shown therein.

In addition to the methods described in the publications cited immediately above, the renal epithelial cells may also be grown on solid or porous microcarrier beads. Examples of suitable microcarrier beads include microporous gelatin and collagen-coated dextran. In this embodiment, the cells can be grown on the beads. Then, the cells can be detached from the beads and be seeded in the RAD. In another embodiment, the cells on the beads can be used in the extracapillary space of a hollow fiber cartridge as opposed to single monolayers along the inner surface of hollow fibers. Thus, a body fluid of a patient could be perfused into a cartridge containing these cells in this formulation for exposure of the patient's fluid to provide amelioration of the cardiorenal syndrome.

The renal epithelial cells may be obtained from a human or non-human animal source. The non-human animal is preferably a mammal. Suitable examples of non-human cells are porcine, rat, dog, mouse, or rabbit tubule cells. Transformed renal epithelial cells may also be used in the present invention. Such cells are described in, for example, U.S. Patent Nos. 6,150,164, 6,410,320, and 5,686,289, incorporated herein by reference.

The body fluid may be blood, plasma, or ultrafiltrate of plasma. Venous blood is particularly preferred. Arterial blood may also be used.

In one embodiment of the invention, the body fluid of the patient is in fluid communication with the renal epithelial cells ex vivo, i.e., outside of the body of the patient. In an alternative embodiment, the body fluid is fluid communication with the renal epithelial cells inside the body of the patient.

In another embodiment, the renal tubule assist device is ex vivo. Alternatively, the renal tubule assist device may be implanted in the patient.

The patient may also be afflicted with chronic renal insufficiency.

An example of a specific embodiment of the present invention is shown in the FIGURE. In this embodiment, venous blood (1) from the patient (20) afflicted with cardiorenal syndrome or hepatorenal syndrome is removed. The removed blood (1) is sent via pump (3) to hemofilter (4). Replacement fluid (2) is added to the venous blood (1) during transport to hemofilter (4). From hemofilter (4), the ultrafiltrate (5) is passed to ultrafiltrate reservoir (6) and the post hemofilter blood (17) is sent to the RAD cartridge (10) after passing through heat exchanger (15). The ultrafiltrate is transferred to the RAD cartridge (10) via pump (7) and heat exchanger (8). Pressure monitor (9) is used to monitor the pressure in the input flows to cartridge (10). Cartridge (10) comprises extracapillary space (11), fiber wall (12), renal epithelial cells (13), and luminal space (14). Post RAD blood (18) is then transferred via pump (19) back to patient (20). The processed ultrafiltrate (16) is collected from the RAD cartridge (10).

Obviously, numerous modifications and variations of the present invention are possible in light of the above teachings. It is therefore to be understood that within the scope of the appended claims, the invention may be practiced otherwise than as specifically described herein.

What is claimed is:

1. A method of treating a patient with cardiorenal syndrome, comprising exposing at least a portion of the body fluid of the patient to renal epithelial cells, outside of the kidney of the patient, whereby the body fluid comes into fluid communication with the renal epithelial cells and is modified by the renal epithelial cells.

2. The method of claim 1, wherein the patient is a mammal.

3. The method of claim 1, wherein the patient is a human.

4. The method of claim 1, wherein the patient is a non-human mammal.

5. The method of claim 1, wherein a portion of the body fluid is removed from the patient, the removed portion of the body fluid comes into fluid communication with the renal epithelial cells and is modified by the renal epithelial cells, and then the body fluid which has been in fluid communication and modified by the renal epithelial cells is returned to the patient.

6. The method of claim 5, wherein the body fluid is blood.

7. The method of claim 5, wherein the body fluid is plasma.

8. The method of claim 5, wherein the body fluid is ultrafiltrate of plasma.

9. The method of claim 1, wherein the body fluid is blood.

10. The method of claim 1, wherein the body fluid is plasma.

11. The method of claim 1, wherein the body fluid is ultrafiltrate of plasma.

12. The method of claim 1, wherein the body fluid comes into fluid communication with the renal epithelial cells ex vivo.

13. The method of claim 1, wherein the body fluid comes into fluid communication with the renal epithelial cells inside the body of the patient.

14. The method of claim 1, wherein the body fluid comes into fluid communication with the renal epithelial cells in a renal tubule assist device.

15. The method of claim 14, wherein the renal tubule assist device is ex vivo.

16. The method of claim 14, wherein the renal tubule assist device is implanted in the patient.

17. The method of claim 1, wherein the patient is also afflicted with chronic renal insufficiency.

18. The method of claim 1, wherein the renal epithelial cells provide and/or catabolize one or more factors which improve cardiac contractility, diminish renal vascular resistance and provide improved native kidney function with increased urine formation.

19. The method of claim 18, wherein the factors are selected from the group consisting of hormone, renalase, vitamin, cytokine and vasoactive compound.

20. The method of claim 19, wherein the vitamin is activated vitamin D.

21. The method of claim 19, wherein the cytokine is selected from the group consisting of IL-1, IL-6, IL-8, IL-10 and G-CSF.

22. The method of claim 19, wherein the vasoactive compound is selected from the group consisting of renin, angiotensin and kallekreins.

23. A method of treating a patient with hepatorenal syndrome, comprising exposing at least a portion of the body fluid of the patient to renal epithelial cells, outside of the kidney of the patient, whereby the body fluid comes into fluid communication with the renal epithelial cells and is modified by the renal epithelial cells.

24. The method of claim 23, wherein the patient is a mammal.

25. The method of claim 23, wherein the patient is a human.

26. The method of claim 23, wherein the patient is a non-human mammal.

27. The method of claim 23, wherein a portion of the body fluid is removed from the patient, the removed portion of the body fluid comes into fluid communication with the renal epithelial cells and is modified by the renal epithelial cells, and then the body fluid which has been in fluid communication and modified by the renal epithelial cells is returned to the patient.

28. The method of claim 27, wherein the body fluid is blood.

29. The method of claim 27, wherein the body fluid is plasma.

30. The method of claim 27, wherein the body fluid is ultrafiltrate of plasma.

31. The method of claim 23, wherein the body fluid is blood.

32. The method of claim 23, wherein the body fluid is plasma.

33. The method of claim 23, wherein the body fluid is ultrafiltrate of plasma.

34. The method of claim 23, wherein the body fluid comes into fluid communication with the renal epithelial cells ex vivo.

35. The method of claim 23, wherein the body fluid comes into fluid communication with the renal epithelial cells inside the body of the patient.

36. The method of claim 23, wherein the body fluid comes into fluid communication with the renal epithelial cells in a renal tubule assist device.

37. The method of claim 36, wherein the renal tubule assist device is ex vivo.

38. The method of claim 36, wherein the renal tubule assist device is implanted in the patient.

39. The method of claim 23, wherein the patient is also afflicted with chronic renal insufficiency.

40. The method of claim 23, wherein the renal epithelial cells provide and/or catabolize one or more factors which improve cardiac contractility, diminish renal vascular resistance and provide improved native kidney function with increased urine formation.

41. The method of claim 40, wherein the factors are selected from the group consisting of hormone, renalase, vitamin, cytokine and vasoactive compound.

42. The method of claim 41, wherein the vitamin is activated vitamin D.

43. The method of claim 41, wherein the cytokine is selected from the group consisting of IL-1, IL-6, IL-8, IL-10 and G-CSF.

44. The method of claim 41, wherein the vasoactive compound is selected from the group consisting of renin, angiotensin and kallekreins.
